(19) United States
(12) Patent Application Publication
(10) Pub. No.: US 2022/0218809 A1
(43) Pub. Date: Jul. 14, 2022

(54) IDO ACTIVITY AS A MARKER OF TUMOR IMMUNE ESCAPE AND IDO INHIBITORS AS A MEANS OF ENHANCING T CELLS RESPONSE TO ANTIGEN SPECIFIC VACCINE

(71) Applicant: Wisconsin Alumni Research Foundation, Madison, WI (US)

(72) Inventors: Douglas G. McNeel, Madison, WI (US); Christopher D. Zahm, Madison, WI (US); Laura Johnson, Madison, WI (US)

(73) Assignee: Wisconsin Alumni Research Foundation, Madison, WI (US)

(21) Appl. No.: 17/291,159

(22) PCT Filed: Nov. 6, 2019
(86) PCT No.: PCT/US2019/060077

§ 371 Date: May 4, 2021

Related U.S. Application Data
(60) Provisional application No. 62/756,157, filed on Nov. 6, 2018.

(51) Int. Cl. A61K 39/00; A61P 35/00; A61K 39/395; A61K 31/4245

(52) U.S. Cl. CPC A61K 39/001193; A61P 35/00; A61K 2039/545; A61K 31/4245; A61K 2039/53; A61K 39/3955

(57) ABSTRACT

The present invention provides compositions and methods of treating prostate cancer using a combination of a DNA vaccine, PD-1 inhibitor and an IDO inhibitor. Further, methods of measuring IDO activity as a way to identify a subpopulation of subjects with prostate cancer that may benefits from the treatment methods described herein are provided.

### CROSS-RELATED APPLICATIONS

This application claims benefit to U.S. provisional application No. 62/756,157 filed on Nov. 6, 2018, the contents of which is incorporated herein by reference in its entirety.

### STATEMENT REGARDING FEDERALLY SPONSORED RESEARCH AND DEVELOPMENT

This invention was made with government support under CA219154 awarded by the National Institutes of Health. The government has certain rights in the invention.

### BACKGROUND

The growth of tumors in an immune competent host suggests that mechanisms of immune avoidance are employed to evade immune-mediated destruction (1). Tumors can acquire or use many different means of immune avoidance, including 1) acquisition of immunosuppressive cell populations such as regulatory T cells, myeloid-derived suppressor cells (MDSC), and tumor-associated macrophages; 2) expression of ligands such as PD-L1 that can interfere with T cell function; 3) loss of expression of immune recognition markers such as MHC class I; and 4) elaboration of cytokines and chemokines that can interfere with immune cell function and recognition.

Indoleamine 2,3 dioxygenase (IDO), an INFy-inducible tryptophan-metabolizing enzyme, is one such factor produced by tumor cells and other cells that has known immunosuppressive properties (2, 3). IDO is the first and rate-limiting catabolic enzyme in the degradation pathway of tryptophan. By cleaving the aromatic indole ring of tryptophan, IDO initiates the production of a variety of tryptophan degradation products, including kynurenines. Tryptophan depletion leads to profound immune-regulatory functions, including inhibiting lymphocyte expansion and recruitment of regulatory T cells and myeloid-derived suppressor cells to the tumor microenvironments (4, 6). Expression of IDO has previously been established as one potential mechanism of resistance to CTLA-4 blockade therapy, spurring the clinical development of IDO inhibitors as potential anti-cancer therapies to combine with T-cell checkpoint inhibitors (7). However, one clinical study that assessed IDO inhibition using epacadostat in combination with PD-1 blockade with pembrolizumab failed to demonstrate improvement over pembrolizumab treatment alone (8).

Prostate cancer is generally considered to be a poorly immunogenic cancer, devoid of large numbers of tumor-infiltrating lymphocytes (TIL), and containing immunosuppressive populations, including MDSC. Unlike many solid tumors, prostate cancer has been relatively refractory to treatment with CTLA-4 or PD-1/PD-L1 targeted therapies alone. On the other hand, IDO expression has been detected in transgenic adenocarcinoma mouse prostate (TRAMP) tumors and was associated with early prostate cancer progression, suggesting that IDO might be a therapeutic target for prostate cancer (9). Likewise, IDO expression has been detected in human prostate cancers (10) and in human prostate cancer undergoing epithelial-mesenchymal transition (11). However the detection of IDO activity, at least as measured by serum kynurenine-to-tryptophan (kyn:trp) ratio, has been debated as a biomarker of prostate cancer detection or progression (12, 13). In addition, it remains unknown whether IDO expression is specifically used by prostate cancer as a mechanism of immune evasion.

There is still a need for cancer therapies that can target tumor cells that evade the immune system.

### SUMMARY OF INVENTION

In one aspect, the disclosure provides a method of reducing or inhibiting proliferation of a prostate cancer cell, killing or inducing apoptosis of a prostate cancer cell, or combinations thereof in a subject having prostate cancer, the method comprising administering the subject at least one indoleamine 2,3 dioxygenase (IDO) inhibitor, at least one PD-1 inhibitor or PD-L1 inhibitor, and a DNA vaccine against prostate cancer, each in an amount effective, in combination, to reduce or inhibit growth of the prostate cancer cell, induce killing or apoptosis of the prostate cancer cell, or a combination thereof.

In another aspect, the present disclosure provides a method of treating a subject having prostate cancer, the method comprising administering at least one indoleamine 2,3 dioxygenase (IDO) inhibitor, at least one PD-1 inhibitor or PD-L1 inhibitor, and a DNA vaccine against prostate cancer, each in an amount effective, in combination, to treat the prostate cancer in the subject.

In yet another aspect, the present disclosure provides a method of eliciting an immune response by a DNA vaccine against a cancer in a subject having received or receiving the DNA vaccine, the method comprising: administering to the subject at least one indoleamine 2,3 dioxygenase (IDO) inhibitor and at least one PD-1 inhibitor or PD-L1 inhibitor, each administered in an effective amount, in combination, such that when the DNA vaccine, the IDO inhibitor, and the PD-1 inhibitor or PD-L1 inhibitor are administered to the subject the immune response elicited is greater than the immune response elicited by the DNA vaccine alone.

The foregoing and other aspects and advantages of the invention will appear from the following description. In the description, reference is made to the accompanying drawings that form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIGS. 1A-1C: Kynurenine:tryptophan ratios are higher in patients with advanced prostate cancer. Sera or plasma samples were evaluated for kynurenine and tryptophan concentrations from normal male volunteer blood donors (n=12), patients with newly diagnosed prostate cancer pre-treatment (n=14), patients with non-castrate, PSA-recurrent non-metastatic (M0) prostate cancer (n=15), castration-resistant, M0 prostate cancer (n=15), and castration-resistant, metastatic prostate cancer (n=16). Shown are the ratios of kynurenine-to-tryptophan for each group (FIG. 1A), and overall with respect to subject age (FIG. 1B) or serum PSA for individuals with prostate cancer (FIG. 1C). Open circles in FIG. 1B are normal male blood donors. For FIG. 1A, *=p<0.05 (Kruskal-Wallis test with Dunn's correction for multiple comparisons). Tests of correlation with age and PSA (FIG. 1B-1C) were made by Pearson test.

FIGS. 1D-1F: Kynurenine concentration are higher in sera of patients with advanced prostate cancer. Sera or plasma samples were evaluated for kynurenine concentrations from normal male volunteer blood donors (n=12), patients with newly diagnosed prostate cancer pre-treatment (n=14), patients with non-castrate, PSA-recurrent non-metastatic (M0) prostate cancer (n=15), castration-resistant, M0 prostate cancer (n=15), and castration-resistant, metastatic prostate cancer (n=16). Shown are the concentrations of kynurenine for each group (FIG. 1D), and overall with respect to subject age (FIG. 1E) or serum PSA for individuals with prostate cancer (FIG. 1F). Open circles in FIG. 1E are normal male blood donors. For FIG. 1D, *=p<0.05 (Kruskal-Wallis test with Dunn's correction for multiple comparisons). Tests of correlation with age and PSA (FIG. 1E-1F) were made by Spearman test.

FIGS. 2A-2F: Patients with prostate cancer treated with an anti-tumor vaccine and/or PD-1 blockade develop increased IDO activity.

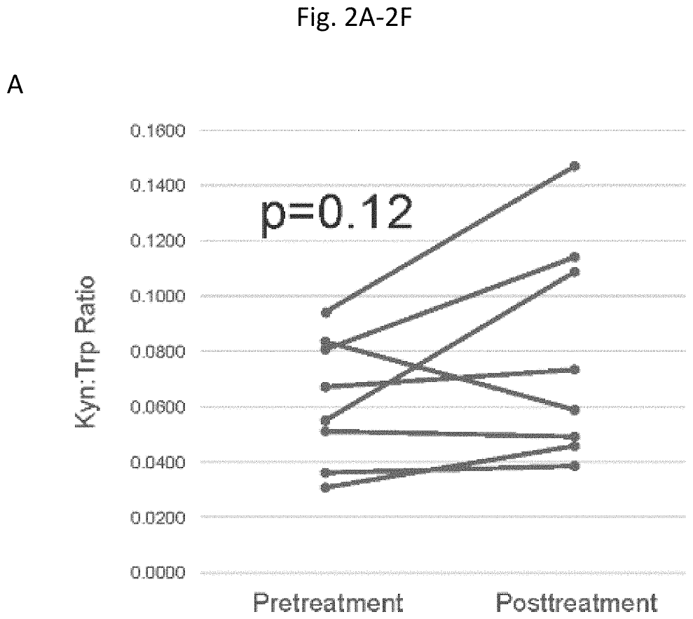

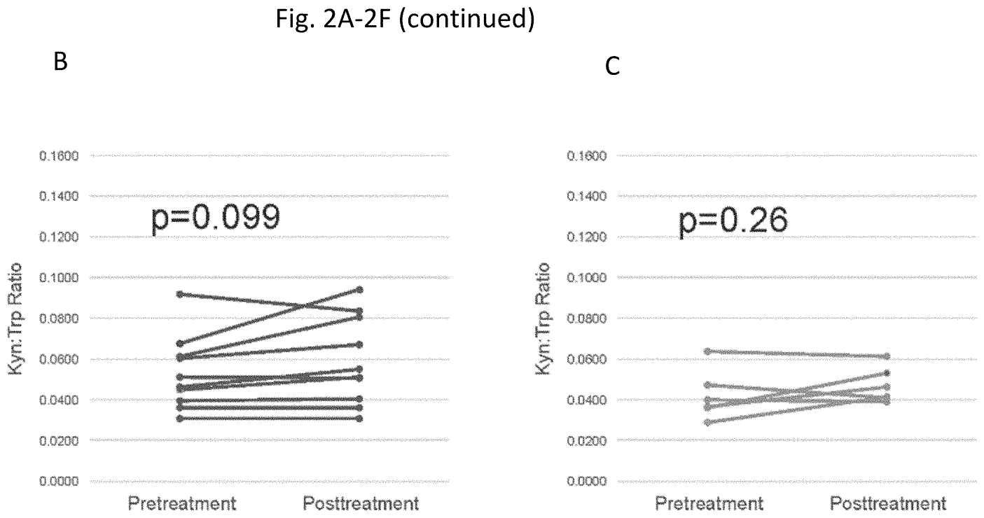

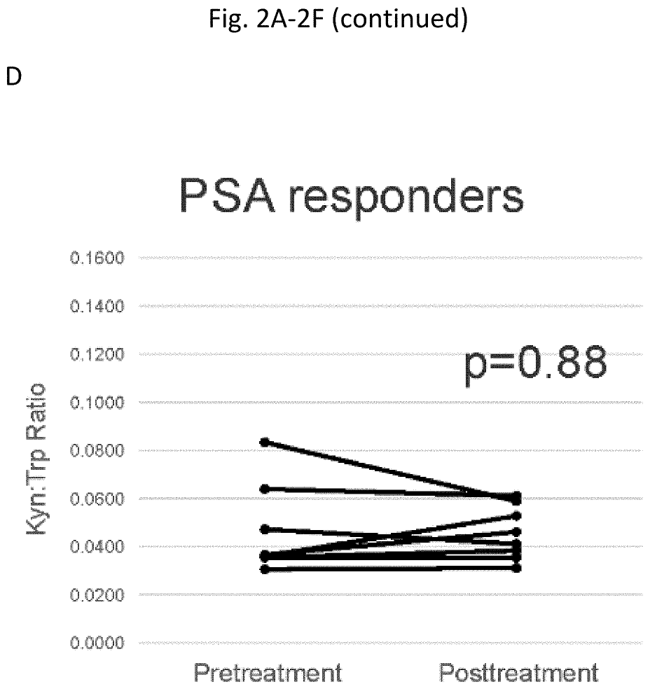

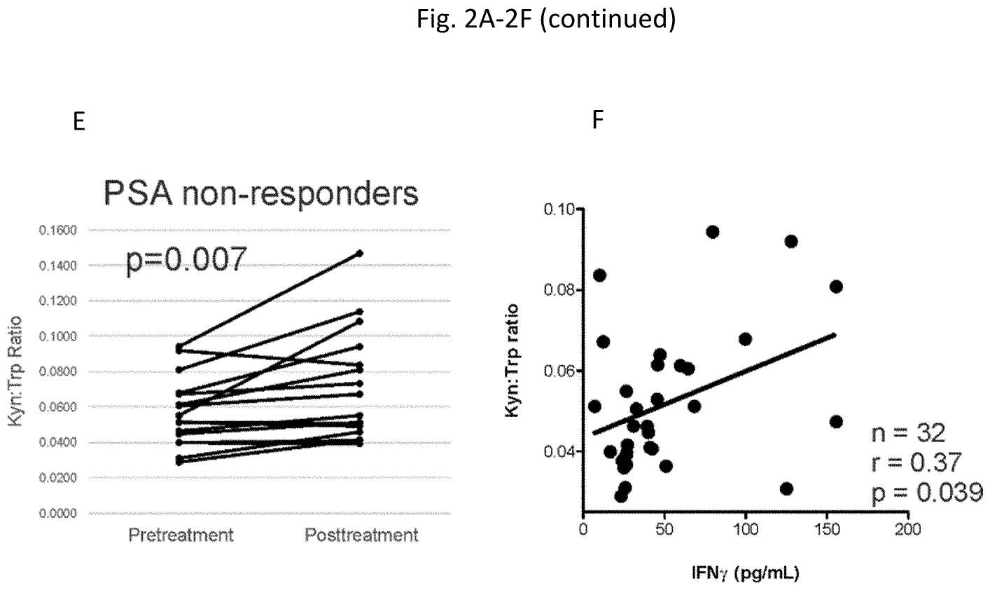

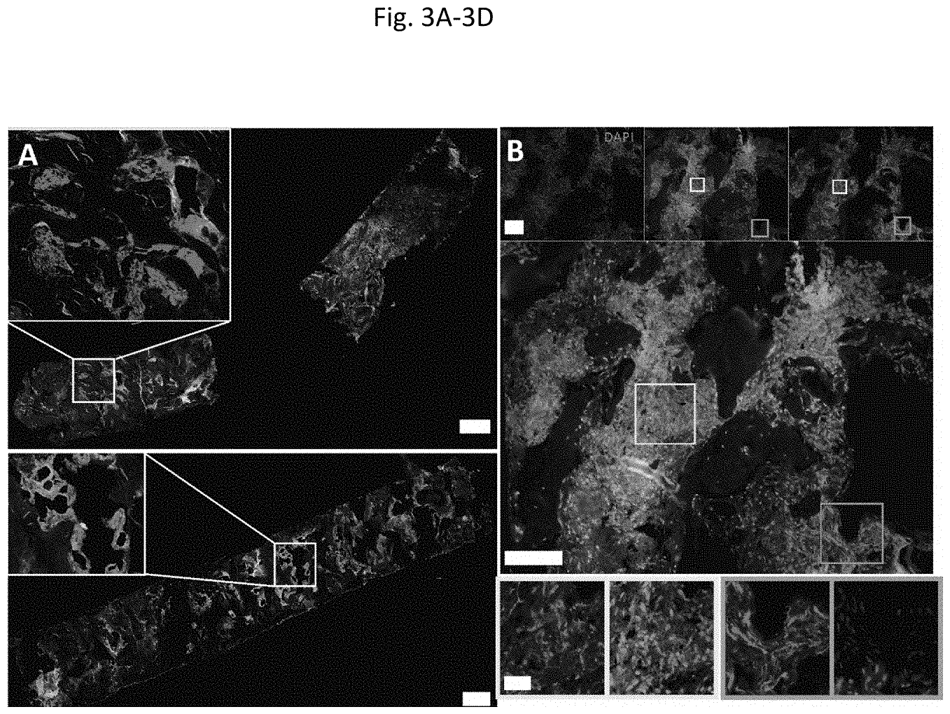

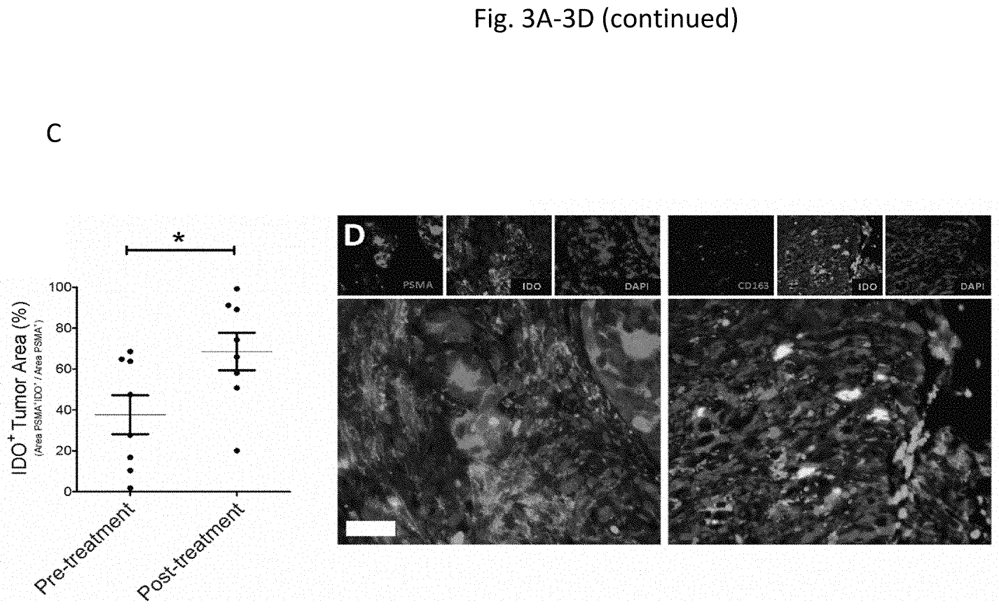

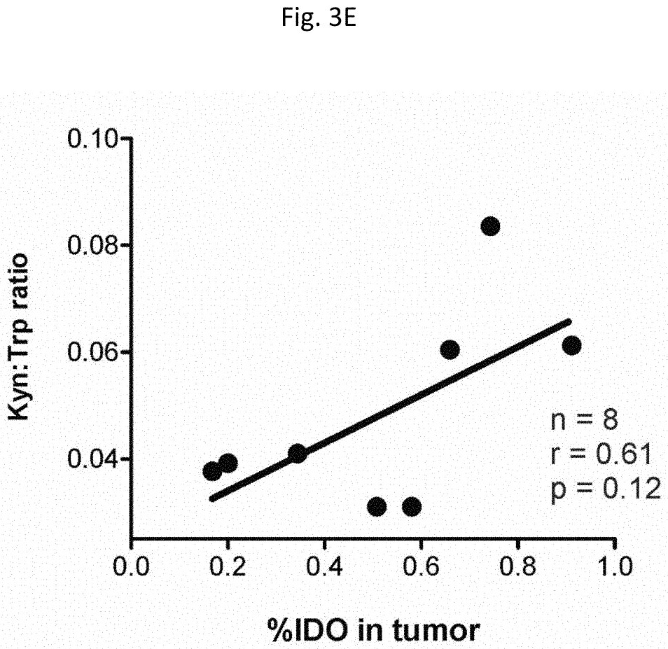

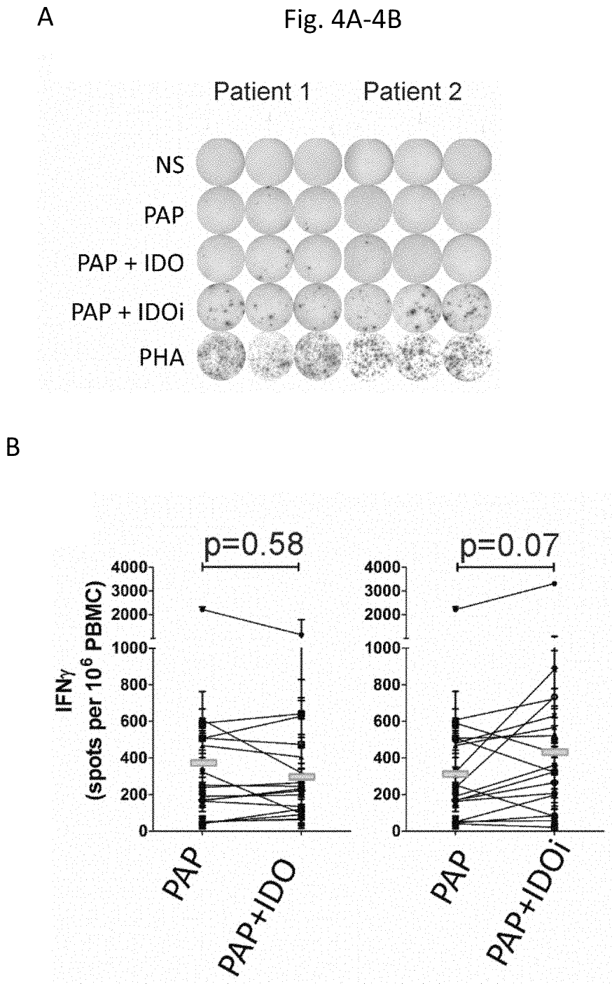

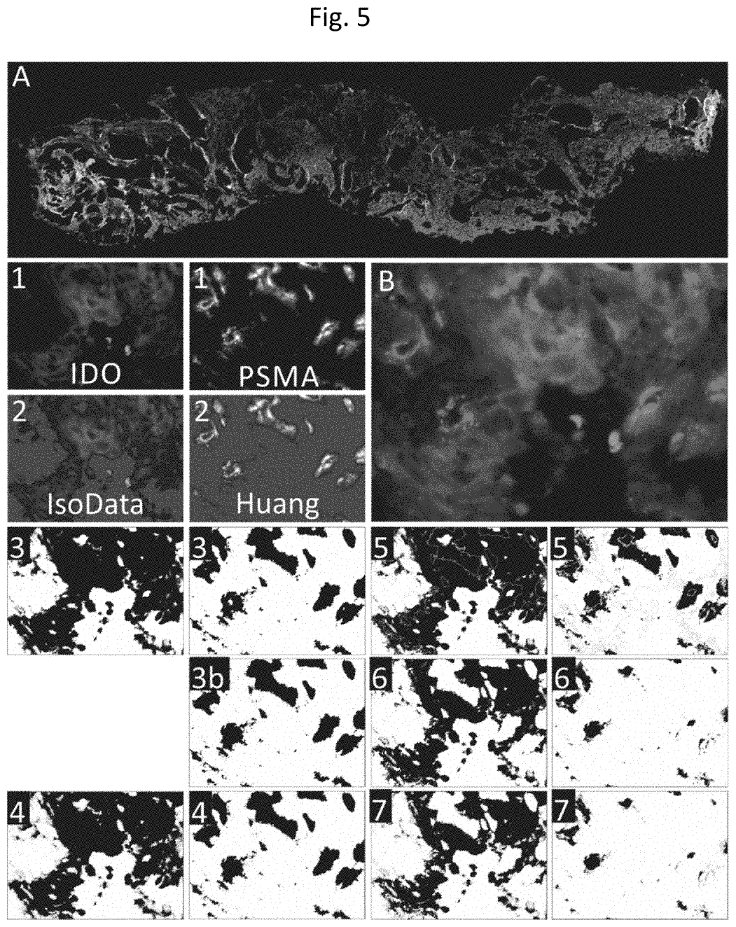

Kyn:trp ratios were evaluated in patients prior to treatment and after 12 weeks treatment with pembrolizumab alone (n=8, FIG. 2A), pTVG-HP DNA vaccine alone (n=10, FIG. 2B), or both agents together (n=6, FIG. 2C). Kyn:trp ratios were evaluated in subsets of patients who experienced any PSA decline over the same 12-week period of treatment (n=8, FIG. 2D), and those who did not have any PSA decline over the same 12-week period of treatment (n=16, FIG. 2E). Comparisons were made with a paired student t test. Kyn:trp ratios were evaluated with respect to serum IFNγ concentration (FIG. 2F). Test of correlation was made by Spearman test.

FIGS. 3A-3D: Patients with prostate cancer treated with PD-1 blockade and/or an anti-tumor vaccine develop increased IDO expression in prostate tumor microenvironment.

Metastatic tissue biopsies obtained pre-treatment and at 12 weeks were obtained from 8 patients and evaluated by immunofluorescence for DAPI, IDO, and prostate-specific membrane antigen (PSMA) expression. FIG. 3A: Shown are entire sections from one individual at baseline (pre) and week 12 (post) demonstrating variable expression in different tumor sections (10× magnification, scale bars=1000 μm). FIG. 3B: Higher magnification (40×, scale bars=200 μm top four images, scale bar=50 μm bottom four images) to demonstrate individual and composite staining with immunofluorescent staining for PSMA (red)/IDO (green)/DAPI (blue). FIG. 3C: Quantification of IDO staining within PSMA+ tumor regions for all samples. FIG. 3D: Immunofluorescent staining of a representative post-treatment tumor sample for PSMA (red)/IDO (green)/DAPI (blue, left) and CD163 (red)/IDO (green)/DAPI (blue, right) (20× magnification, scale bar=100 μm) Statistical comparison was made using a paired Wilcoxon signed rank test.

FIG. 3E: Association of tumor expression of IDO and serum kyn:trp ratio. Quantitative imaging performed as in FIGS. 3A-3D was used to determine the % IDO staining within tumor regions. These values are shown in relation to serum kyn:trp ratios obtained from the same individuals at the same time points (n=8). Test of correlation was made by Spearman test.

FIGS. 4A-4B: IDO activity is associated with decreased vaccine antigen-specific T-cell function. Peripheral blood mononuclear cells (PBMC) were obtained from patients treated with vaccine and pembrolizumab (after 12 weeks of treatment). PBMC were cultured in the presence of an overlapping peptide library derived from the PAP vaccine antigen (PAP), media alone, or phytohemaglutinin (PHA) as a positive control. Cells were also cultured with antigen in the presence of IDO enzyme (IDO) or the IDO inhibitor 1-methyltryptophan (IDOi). IFNγ-secreting T cells were detected by ELISPOT. Shown are representative ELISPOTs from two subjects (FIG. 4A), and cumulative data from 22 subjects evaluated (FIG. 4B). Comparisons are made with a two-sided paired t test.

FIG. 5: Methods for quantifying IDO staining within tumor regions. Whole FFPE tumor biopsies were stained with IDO, PSMA, and DAPI as described in the Methods section. Whole section mosaic images were obtained on the Leica DMi8 at 10× (Panels A-B). Using the image in panel B, steps 1-7 depict the image processing steps taken to quantify the area of IDO expression as a percent of area of PSMA. First, a threshold was determined for the original greyscale images using the ImageJ built-in IsoData algorithm for IDO and Huang algorithm for PSMA (Steps 1-2). The threshold was applied, and the image converted into a binary mask. Because PSMA is a membrane stain, for that protein the conversion was followed by the “fills holes” ImageJ function (Steps 3-3b). Once the binary images were created, a selection was made using the ImageJ built-in function (edit/selection/create selection) and the area quantified using the measure function (process/measure) to give total tumor (AT) and IDO-expressing (AI) areas (Step 4). To calculate the percentage of AT that overlapped with AI, the selection of IDO was applied to the mask of PSMA, filled white, and the remaining tissue area measured as described above. The process was repeated for PSMA over IDO (Steps 5-7).

### DETAILED DESCRIPTION OF THE INVENTION

Before the present invention is described, it is understood that this invention is not limited to the particular methodology, protocols, and reagents described, as these may vary. It is also to be understood that the terminology used herein is for describing particular embodiments only, and is not intended to limit the scope of the present invention, which will be limited only by the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies that are reported in the publications that might be used in connection with the invention.

The present disclosure demonstrates results showing that sera from patients with different stages of prostate cancer had IDO activity, as assessed by serum kyn:trp ratios, increased with stage of disease. This activity, and IDO expression in tumors, was markedly induced following treatment with an anti-tumor vaccine and/or PD-1 blockade. IDO activity was found to suppress the function of vaccine-induced T cells, and to be highest in patients who did not demonstrate benefit from immunotherapy (i.e., DNA vaccine, PD-1 inhibitor, or combination). IDO was found as a mechanism of resistance to prostate cancer directed immunotherapy and that changes in IDO activity may be a biomarker of immune response elicited with immunotherapy. IDO activity was increased in patients with more advanced prostate cancer. This activity, and IDO expression as detected immunohistochemically, increased following treatment with either a DNA vaccine encoding the prostatic acid phosphatase (PAP) tumor antigen or PD-1 blockade with pembrolizumab. Increased activity was associated with lack of PSA response to treatment. IDO blunted T-cell response, as measured by IFNγ release, to the vaccine target antigen, and the T cell response could be recovered in the presence of an IDO inhibitor. Thus, the present invention provides methods of treating prostate cancer, including methods of treating prostate cancer in patients that did not benefit from immunotherapy with a PD-1 inhibitor, DNA vaccine or combination thereof by use of the combination of at least one IDO inhibitor, at least one PD-1 inhibitor and a DNA vaccine against prostate cancer.

While the present disclosure is directed to the treatment of prostate cancer, it is understood by one skilled in the art the combination therapy described herein may be used for other cancers, such as solid cancer, that may be treated with DNA vaccines to augment the immune response to the cancer. Suitable cancers include, for example, solid tumors, including, but not limited to, breast cancer, prostate cancer, cervical cancer, ovarian cancer, pancreatic cancer, glioblastoma, melanoma, renal cell carcinoma, melanoma, colon cancer, colorectal cancer, sarcoma, kidney cancer, for example, those summariezed in “Cancer DNA vaccines: current preclinical and clinical developments and future perspectives” Lopes et al. Journal of Experimental and Clinical Cancer Research” 38, 146 (2019), the contents of which are incorporated by reference in its entirety. One skilled in the art would readily be able to exend the teachings herein to other known solid cancers using cancer specific DNA vaccines.

While IDO inhibitors have been used in combination with vaccine treatments and in combination with T-cell checkpoint inhibitors, recent clinical trials showed no benefit to IDO inhibition with PD-1 blockade. Thus, the present methods provide a surprising result that the combination of an IDO inhibitor, at least one PD-1 inhibitor and a DNA vaccine against prostate cancer can activate a T-cell response against the prostate cancer cells, which leads to the treatment of the prostate cancer.

In one aspect, the present disclosure provides a method of reducing or inhibiting proliferation of a prostate cancer cell, increased killing or apoptosis of a cancer cell in a subject having prostate cancer, or combinations thereof in a subject having cancer, specifically prostate cancer, the method comprising administering at least one indoleamine 2,3 dioxygenase (IDO) inhibitor, at least one PD-1 inhibitor or PD-L1 inhibitor, and a DNA vaccine against prostate cancer each in an amount effective, in combination, to reduce or inhibit proliferation of the prostate cancer cell, increase killing or apoptosis of the cancer cell, or combinations thereof in the subject.

In some examples, the subject is selected by (a) obtaining a sample from the subject and (b) detecting an increased level of IDO within the sample from the subject as compared to a control, wherein the subject with an increased IDO level is selected to be administered the method described herein. Suitable methods of detecting IDO activity are known in the art and described herein. For example, the increased level of IDO may be detected: (a) in a tumor sample of the subject by immunohistochemical staining; or (b) in serum sample of the subject by determining the kynurenine-to-tryptophan (kyn:trp) ratio within the serum sample.

In another aspect, the disclosure provides a methods of treating or ameliorating prostate cancer in a patient, the method comprising administering at least one indoleamine 2,3 dioxygenase (IDO) inhibitor, at least one PD-1 inhibitor, and a DNA vaccine against prostate cancer each in an amount effective, in combination, to treat the prostate cancer in the patient.

The term “contacting” or “exposing,” as used herein refers to bringing the disclosed agent or composition and a cancer cell, cancer/tumor microenvironment or other biological entity together in such a manner that the agent(s) and/or composition affect the activity of the prostate cancer cell or tumor microenvironment to reduce or inhibit the prostate cancer cell proliferation or growth or induce cancer cell killing and apoptosis either directly; i.e., by interacting with the cell itself, or indirectly; i.e., by interacting with another molecule, co-factor, factor, protein or nearby cell that results in the effect on the cell. For example, the contacting or exposing to the agent may be to the prostate cancer or to the cancer microenvironment, including effector cells within the area of the cancer, resulting in the ability to treat the cancer. For example, the IDO inhibitor may affect the activity of a nearby effector cell, for example, myeloid cells, within the tumor microenvironment.

As used herein “subject” or “patient” refers to mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. The term “subject” does not denote a particular age or sex. In one specific embodiment, a subject is a mammal, preferably a human.

In one preferred example, the prostate cancer cell is present is a subject having prostate cancer, wherein the subject has been previously treated with a DNA vaccine against prostate cancer with or without a PD-1 inhibitor or PD-L1 inhibitor, and in which the prior treatment was not effective in treating the prostate cancer.

In another preferred example, the prostate cancer is defined by an increase in IDO expression within the tumor microenvironment as compared to a control cell. In other words, the subject with prostate cancer being treated by the methods described herein is a subject in which the prostate cancer subject has increased IDO expression either as detected systemically (e.g., from a serum sample) or in the tumor microenvironment (e.g., tumor sample or tumor biopsy).

For purposes of the present invention, “treating” or “treatment” describes the management and care of a subject for the purpose of combating the disease, condition, or disorder. Treating includes the administration of the combination of agents of present invention to prevent the onset of the symptoms or complications associated with cancer, alleviating the symptoms or complications of cancer, or eliminating the cancer. Treating also encompasses therapeutic and palliative treatment.

The term “treating” can be characterized by one or more of the following: (a) the reducing, slowing or inhibiting the growth or proliferation of cancer cells or tumor cells (e.g., cancers or tumors), including reducing slowing or inhibiting the growth or proliferation of prostate cancer cells; (b) preventing the further growth or proliferation of prostate cancer cells; (c) reducing or preventing the metastasis of prostate cancer cells within a patient, (d) killing or inducing apoptosis of cancer cells, and (d) reducing or ameliorating at least one symptom of prostate cancer. Symptoms of prostate cancer are known in the art and include, but are not limited to, urinary obstruction, pelvic pain due to local invasion and metastatic symptoms, including anemia and bone pain, urinary hesitancy, urinary dribbling, urinary retention, pain with urination, pain with ejaculation, lower back pain, pain with bowel movement, excessive urination at night, incontinence, bone tenderness, hematuria, abdominal pain, anemia, weight loss, and lethargy. In some embodiments, the optimum effective amounts can be readily determined by one of ordinary skill in the art using routine experimentation.

In one embodiment, the term treating is characterized by a reduction in the number of prostate cancer cells in a subject. In another embodiment, the treatment can result in cell-cycle inhibition of tumor cells (i.e., cytostasis). In another embodiment, treating is characterized by cancer cell killing and/or apoptosis.

The terms “cancer,” “tumor” or “malignancy” are used throughout this description interchangeably and refer to the diseases of abnormal cell growth. The present methods are for the treatment and amelioration of prostate cancer.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

As used herein, the terms “effective treatment” refers to the treatment producing a beneficial effect, e.g., yield a desired therapeutic response without undue adverse side effects such as toxicity, irritation, or allergic response. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth. The terms “effective amount” or “therapeutically effective amount” refer to an amount sufficient to effect beneficial or desirable biological or clinical results. That result can be reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system. It is understood that the effective amount may differ from patient to patient and drug/composition to drug/composition, and the ability to select the proper effective amount would be within the level of skill in the art.

In a particular embodiment, the subject may suffer from prostate cancer.

DNA vaccines that target prostate cancer are known in the art. Suitable vaccines for use in the present methods include, for example, a recombinant DNA vaccine that encodes the androgen receptor or fragments thereof or a peptide vaccine comprising a polypeptide androgen receptor or fragments thereof. Suitable recombinant DNA vaccines are disclosed in U.S. Pat. Nos. 7,910,565, 8,513,210 and 8,962,590, entitled “Prostate cancer vaccine” each of which is incorporated herein by reference in its entirety. In some embodiments, the DNA vaccine comprises pTVG-AR (pTVG-AR or pTVG-ARLBD refer to the same vector and both designations are used interchangeably herein). The pTVG-AR vector comprises the coding sequence for the ligand-binding domain of the human androgen receptor gene inserted into the pTVG4 vector to create the immunization vector pTVG-AR, as disclosed in U.S. Pat. No. 7,910,565. Other suitable DNA vaccines encode native or modified SSX2 as described in Smith et al. 2011 (Vaccines targeting the cancer-testis antigen SSX-2 elicit HLA-A2 epitopes specific cytolytic T cells. J. Immunother 2011:34:569-80) and Smith et al. 2014 (DNA vaccines encoding altered peptide ligands for SSX2 enhance epitope-specific CD8+ T cell immune responses. Vaccine 2014:32:1707-15), each of which is incorporated herein by reference in its entirety. Other suitable prostate cancer vaccines include vaccines then encode prostatic acid phosphatase (PAP), for example, but not limited to, U.S. Pat. No. 7,179,797 and U.S. application Ser. No. 11/615,778 entitled “Methods and compositions for treating prostate cancer using DNA vaccines” and U.S. application Ser. No. 15/430,012 entitled “Cancer Therapy”, each of which is incorporated herein by reference in its entirety. Suitable dosages and schedules for administering the DNA vaccine would be readily understood by one skilled in the art, and would depend on the patient, the DNA vaccine, factors about the patient (age, weight, etc.), route of administration, and the additional drugs administered in combination. For example, by not limited to, the DNA vaccine may be administered at about 10 μg to −1 mg per dose, e.g., 100 μg) (and in some cases higher amounts), administered by a standard schedule over a period of months or years.

Suitable PD-1 inhibitors for use in the kits and methods described herein are known in the art and include, but are not limited to, for example, anti-PD-1 antibodies. Preferred anti-PD-1 antibodies are anti-PD-1 monoclonal antibodies. Suitable anti-PD-1 antibodies include, but are not limited to, for example, pembrolizumab (Keytruda, Merck), nivolumab (Opdivo, BMS-936559, Bristol-Myers Squibb), cemiplimab (Libtayo, Regeneron Pharmaceuticals Inc., Sanofi), avelumab (Bavencio, Pfizer), durvalumab (Imfinzi, AstraZeneca), atezolizumab (Tecentriq, Genentech), pidilizumab (Cure Tech), AMP-224, (GlaxoSmithKline), AMP-514 (GlaxoSmithKline), PDR001 (Novartis), among others. Other suitable anti-PD-1 antagonists include, but are not limited to, for example; MEDI0680 (Medlmmune/AstraZeneca); MEDI4736 (Medlmmune/AstraZeneca); MPDL3280A (Genentech/Roche), MSB0010718C (EMD Serono), among others.

Suitable PD-L1 inhibitors are known in the art and include, but are not limited to, for example, atezolizumab (Roche), avelumab (Merck Serono and Pfizer), durvalumab (AstraZeneca), BMS-936559 (Bristol-Myers Squibb), CK-301 (Checkpoint Therapeutics), among others.

For example, in some embodiments, the monoclonal antibody is pembrolizumab. Pembrolizumab is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. Accordingly, in some embodiments the same dose and schedule is used as has been used for the approved melanoma indication. For example, in some embodiments pembrolizumab is administered at 2 mg/kg (rounded to the nearest 50 mg) as an intravenous infusion over 30 minutes every 3 weeks (up to four maximum doses). Pembrolizumab is available in single-use vials, consisting of 50 mg lyophilized powder for injection. It is prepared by addition of 2.3 mL of sterile water for injection, USP, to the vial to prepare a 25 mg/mL solution. In some embodiments, it is transferred to an IV bag containing 0.9% sodium chloride injection, USP such that the final concentration of the diluted solution is between 1 mg/mL and 10 mg/mL. Accordingly, in some embodiments it is administered as an intravenous infusion, e.g., over 30 minutes using an IV line containing a sterile, non-pyrogenic, low-protein binding 0.2 μm to 5 μm in-line or add-on filter.

In some embodiments, the monoclonal antibody is nivolumab. Nivolumab is a human IgG4 anti-PD-1 monoclonal antibody that acts as an immunomodulator by blocking ligand activation of the programmed cell death 1 (PD-1) receptor on activated T cells. In particular, nivolumab acts by blocking a negative regulator of T-cell activation and response, thus allowing the immune system to attack the tumor. That is, nivolumab blocks PD-L1 from binding to PD-1, allowing the T cell to function in tumor attack.

Suitable IDO inhibitors for use in the methods described herein are known in the art and include, but are not limited to, for example, 1-methyltryptophan (1-MT), epacadostat (INCB24360), navoximod (GDC-0919), Indoximod (NLG-8189), INCB024360, BMS-986205, NLG919, PF-06840003, or 8-nitrotryptanthrin.

In some embodiments, the combination of agents of the present invention is used for treatment in addition to standard treatment options, for example surgery and radiation therapy.

As used herein, the terms “administering” and “administration” refer to any method of providing the treatment to the patient, for example, any method of providing a pharmaceutical preparation to a subject. Such methods are well known to those skilled in the art and include, but are not limited to, oral administration, transdermal administration, administration by parenteral administration, including injectable such as intravenous administration, intra-arterial administration, intramuscular administration, intradermal administration, intrathecal administration and subcutaneous administration, rectal administration, sublingual administration, buccal administration, among others. Administration can be continuous or intermittent. In various aspects, a preparation or combination of agents can be administered therapeutically; that is, administered to treat an existing disease or condition.

Administration may be simultaneously (e.g., at the same time) or sequentially. In some embodiments, the DNA vaccine is administered according to a proper vaccine and booster schedule and the IDO inhibitor and PD-1 inhibitor are administered daily or weekly depending on the dosage and formulations.

The term “simultaneous administration,” as used herein, means that the agents (e.g., the IDO inhibitor and PD-1 inhibitor) are administered with a time separation of no more than about 1 day (e.g., each component is administered within 24 hours, for example, within 2, 4, 6, 8, 10, or 12 hours, or, in other examples, within 60, 50, 40, 30, 20, or 10 minutes). When the agents are administered simultaneously, the agents (e.g., the IDO inhibitor and the PD-1 inhibitor) may be contained in the same composition or in separate compositions (e.g., the IDO inhibitor is contained in one composition and the PD-1 inhibitor is contained in another composition).

In some embodiments, the agents are administered sequentially or intermittently. The term “sequential administration” as used herein means that the agents or compositions are administered with a time separation of more than about 1 day. In one embodiment, the DNA vaccine is administered first followed by daily or weekly administration of the IDO inhibitor and the PD-1 inhibitor that are administered each daily or weekly. Suitably, when administered sequentially, for example, the IDO inhibitor may be administered first followed by administration of the PD-1 inhibitor or the PD-1 inhibitor may be administered first followed by administration of the IDO inhibitor. The time between the administration of the IDO inhibitor and PD-1 inhibitor can be adjusted for maximum efficacy, and may be in the order of minutes, hours, days or weeks. In some embodiments, the agents are administered intermittently. The term “intermittent administration” as used herein refers to administration that does not occur daily, for example, administration of a PD-1 inhibitor that occurs weekly, biweekly, etc. In some embodiments, the IDO inhibitor may be administered daily or continuously and the PD-1 inhibitor and DNA vaccine are administered intermittently.

The pharmaceutical compositions described herein may further include a pharmaceutically acceptable carrier. The term “pharmaceutically acceptable carrier” refers any carrier, diluent or excipient that is compatible with the other ingredients of the formulation and not deleterious to the recipient.

The at least one PD-1 inhibitor or at least one IDO inhibitor may preferably be administered each with a pharmaceutically acceptable carrier selected on the basis of the selected route of administration and standard pharmaceutical practice for each inhibitor. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, injectable solutions, troches, suppositories, or suspensions. For antibodies, suitable dosages forms are normally solutions.

For oral administration, the active ingredient may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents.

For parenteral administration, the active agent may be mixed with a suitable carrier or diluent such as water, an oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

The pharmaceutical composition is preferably in unit dosage form. In such form, the preparation is divided into unit doses containing appropriate quantities of the active component.

It will be appreciated that appropriate dosages of the IDO inhibitor or PD-1 inhibitor, and compositions comprising an IDO inhibitor or PD-1 inhibitor, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, or materials used in combination, and the age, sex, weight, condition, general health, and prior medical history of the patient. The amount of compound and route of administration will ultimately be at the discretion of the physician. Administration in vivo can be effected in one dose, continuously or intermittently (e.g., in divided doses at appropriate intervals) throughout the course of treatment.

Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In some embodiments, the subject is a human having prostate cancer that was non-responsive to the treatment by the combination of PD-1 inhibitor and DNA vaccine.

In another aspect, the present disclosure provides a method of eliciting an immune response by a DNA vaccine against a cancer in a subject having received or receiving the DNA vaccine, the method comprising: administering to the subject at least one indoleamine 2,3 dioxygenase (IDO) inhibitor and at least one PD-1 inhibitor, each administered in an effective amount, in combination, such that when the DNA vaccine, the IDO inhibitor, and the PD-1 inhibitor are administered to the subject the immune response elicited is greater than the immune response elicited by the DNA vaccine alone. In some embodiments, the immune response is a cell-mediated immune response. In a preferred embodiment, the cell-mediated response is a T cell response, for example, a CD8+ T cell response. In some embodiments, the immune response is an immune-mediated anti-tumor response.

In some embodiments, the methods increase the anti-tumor response in a subject, the method comprising administering at least one IDO inhibitor, at least one PD-1 inhibitor and a DNA vaccine against prostate cancer to the subject in need of an anti-tumor response (for example, a subject having prostate cancer). Such increase in the anti-tumor response may be demonstrated by an increased anti-tumor response in an animal model of the tumor as compared with the animal model without treatment.

In one embodiment, the present disclosure provides a method of eliciting, enhancing or improving an anti-tumor response in a subject in need thereof. An “improved immune-mediated anti-tumor response” means an increase in the ability of one or more immune cells to recognize tumor cells. In some instances, the improved immune-mediated anti-tumor response results in an increased ability of one or more immune cells to target/recognize and kill cancer cells (e.g., CD8+ T cells). An improved immune-mediated anti-tumor response may be seen as a reduction in the number of cancer cells, inhibiting, retarding or slowing the growth of cancer cells, inhibiting, retarding or slowing the metastasis of cancer cells, increased infiltration of cytotoxic T cells into the tumor, or decreased inhibition of immune population within the tumor microenvironment.

In some embodiments, the anti-tumor response is improved over the response to the DNA vaccine alone or the DNA vaccine in combination with a PD-1 inhibitor only. In some embodiments, the anti-tumor response is a cell-mediated immune response. In some embodiments, the cell-mediated response is an activated T cell response, e.g., CD8+ T cells.

An “improved immune-mediated anti-tumor response” means an increase in the ability of one or more immune cells to recognize tumor cells. In some instances, the improved immune-mediated anti-tumor response results in an increased ability of one or more immune cells to target/recognize and kill cancer cells (e.g., CD8+ T cells). An improved immune-mediated anti-tumor response may be seen as a reduction in the number of cancer cells, inhibiting, retarding or slowing the growth of cancer cells, inhibiting, retarding or slowing the metastasis of cancer cells, increased infiltration of cytotoxic T cells into the tumor, or decreased inhibition of immune population within the tumor microenvironment.

The terms “cell-mediated immune response” or “cell-mediated immunity” refer to an immune response mediated by immune cells and does not involve antibodies (humoral immune response). Specifically, cell-mediated immune response includes antigen-specific cytotoxic T-lymphocytes (CD8+ T cells) or activation of phagocytes. Phagocytes include white blood cells such as neutrophils, monocytes, macrophages, mast cells, and dendritic cells. In a preferred embodiment, the cell-mediated immune response is a cytotoxic T cell response or CD8+ T cell response.

Not to be bound by any theory, but the methods of the present invention provide a method of inducing T-cell activation against cancer cells, e.g., prostate cancer cells using a DNA vaccine (which causes IFNy release) in combination with PD-1 blockade (to block the negative impact of PD-L1 which is increased following IFNγ exposure) and with IDO inhibition (which is also increased following IFNy exposure).

In another embodiment, the present disclosure provides a method of measuring IDO activity (by serum kynurenine and tryptophan levels) within a sample. A higher level of IDO activity (as compared to a control) is a measure of response to immunotherapy (e.g., DNA vaccine) and a way to identify a subpopulation of subjects with prostate cancer that may benefit from the treatment methods described herein, for example, in some embodiments, subjects having castrate-resistant or immunotherapy-resistant cancer.

In another embodiment, the disclosure provides that detecting increased IDO activity is in patients having more advanced prostate cancer (e.g., castrate-resistant or immunotherapy resistant prostate cancer), and this activity is augmented following prostate tumor-directed immunotherapy. Kynurenine and tryptophan concentrations in peripheral blood demonstrated increased IDO levels systemically. Further, increased IDO expression was also detected in prostate tumor tissues. The observation that expression was increased primarily in patients who did not have evidence of anti-tumor effect, and that IDO activity decreases the effector function of vaccine-induced T cells, suggests that it is a specific mechanism of immune resistance in prostate cancer. Thus, a patient who has increased IDO inhibition can be treated with a combination of IDO inhibitor, a DNA vaccine and a PD-1 inhibitor, to increase the anti-tumor effect of the immunotherapies.

Kits

In some embodiments, kits for carrying out the methods described herein are provided. The kits provided may contain the necessary components with which to carry out one or more of the above-noted methods. In one embodiment, a kit for treating a subject having prostate cancer is provided. The kit comprises at least one indoleamine 2,3 dioxygenase (IDO) inhibitor, at least one PD-1 inhibitor, and a DNA vaccine against prostate cancer and instructions for use. In one embodiment, the kit may comprise a pharmaceutical composition comprising the IDO inhibitor and a pharmaceutical composition comprising at least one PD-1 inhibitor and a vaccine composition.

Another embodiment provides a kit for detecting prostate cancer cells in a subject in which the methods described herein of using an IDO inhibitor, PD-1 inhibitor and DNA vaccine may be beneficial.

The kit comprises means for measuring IDO activity by serum kynurenine and tryptophan levels within a prostate cancer sample. High IDO levels as compared with a control can serve to stratify prostate cancer as being responsive to immunotherapy treatment as described herein (e.g., DNA vaccine in combination with a PD-1 inhibitor and an IDO inhibitor).

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The present invention has been described in terms of one or more preferred embodiments, and it should be appreciated that many equivalents, alternatives, variations, and modifications, aside from those expressly stated, are possible and within the scope of the invention.

### EXAMPLES

The invention will be more fully understood upon consideration of the following non-limiting examples.

### Example 1

### Increased IDO Activity in Prostate Cancer

This Example demonstrates that IDO activity was increased in patients with more advanced prostate cancer. This activity, and IDO expression as detected immunohistochemically, increased following treatment with either a DNA vaccine encoding the prostatic acid phosphatase (PAP) tumor antigen or PD-1 blockade with pembrolizumab. Increased IDO activity after treatment was associated with the absence of clinical effect, as assessed by lack of PSA decline following treatment. IDO inhibition tended to increase antigen-specific T-cell response, as measured by IFNy release, to the vaccine target antigen following in vitro stimulation of peripheral blood cells. Blood samples from normal male blood donors (n=12) and patients with different stages of prostate cancer (n=89), including patients with metastatic, castration-resistant prostate cancer treated with a DNA vaccine and/or pembrolizumab, were evaluated for IDO activity by kynurenine and tryptophan levels. Metastatic tissue biopsies obtained pre- and post-treatment were evaluated for IDO expression. IDO activity on vaccine-induced T-cell function was assessed by ELISPOT.

These findings suggest that IDO expression is a mechanism of immune evasion used by prostate cancer cells, and that using T-cell based immune strategies against prostate cancer should incorporate IDO inhibition. IDO activity can also be used as a serum biomarker of immune response to prostate tumors that are elicited with DNA vaccine treatment and indicative of the need for additional treatment with an IDO inhibitor.

Materials and Methods:

Patient and Sample Populations:

Sera or plasma and peripheral blood mononuclear cells (PBMC), cryopreserved at −80° C. or in liquid nitrogen, respectively, were used for these studies. These samples were collected from men without prostate cancer (n=12), patients with newly diagnosed prostate cancer (n=14), patients with biochemically recurrent (rising PSA), non-metastatic prostate cancer that were either non-castrate (n=15), or castration-resistant (n=15), and from men with castration-resistant metastatic prostate cancer (n=16). Samples were collected under University of Wisconsin IRB-approved protocols, and all patients gave written, informed consent for remaining samples to be used for research.

Tryptophan and Kynurenine Analysis:

Tryptophan and kynurenine concentrations were measured directly in serum samples using a clinically validated LC/MS method. All analysis was performed by Worldwide Clinical Trials (Morrisville, N.C.) by personnel blinded to the sample source.

Enzyme-Linked Immunosorbent Assay (ELISA) for Serum IFNγ:

Sera samples were evaluated for IFNγ concentration by capture ELISA using standard methods as previously described (19). Antibodies included an anti-human IFNγ capture antibody (BD Biosciences, San Jose, Calif. #554550) and biotinylated anti-human IFNγ detection antibody (BD Biosciences #551221).

Immunohistochemistry:

Formalin-fixed paraffin-embedded (FFPE) tumor biopsies were stained for IDO, PSMA, or CD163 expression using standard immunofluorescent (IF) techniques. Briefly, slides were heated at 80° C. for 20 min, deparaffinizied, and antigens retrieved using DIVA Decloaker (Biocare Medical, DV2004, Pacheco, Calif.) at 99° C. for 30 min. IDO was detected with primary antibody (Biocare Medical, ACI 3210 B) diluted 1:100 in Renoir Red diluent (Biocare Medical, PD904) followed by an AlexaFluor 488 labeled anti-mouse secondary antibody (Cell Signaling, 4408S, Danvers, Mass.) and subsequently mounted in ProLong Gold Antifade Reagent with DAPI (Cell Signaling, 8961S). PSMA and CD163 were detected with primary antibodies (12815S [1:100] and 934985 [1:500], Cell Signaling) diluted in Van Gogh Diluent (Biocare Medical, PD902 L) labeled with anti-rabbit AlexaFluor 555 secondary (4413S [1:500], Cell Signaling) and mounted in ProLong Gold Antifade Reagent with DAPI. For dual staining, primary and secondary antibodies were combined and co-stained in the Van Gogh diluent. Imaging was conducted on a Leica DMi8 and images processed in the Fiji package of ImageJ (20). The contrast, brightness and color balance were optimized evenly across all areas of each image and for all images.

Image Processing:

Whole FFPE tumor biopsies were stained with IDO/PSMA or IDO/CD163 and DAPI as described above. Whole section mosaic images were obtained on the Leica DMi8 at 10× (FIG. 5). A threshold was then determined for the original greyscale images using the ImageJ built-in IsoData algorithm for IDO and Huang algorithm for PSMA. The threshold was applied, and the image converted into a binary mask. Because PSMA is a membrane stain, the conversion for that protein was followed by the “fills holes” ImageJ function. Once the binary images were created a selection was made using the ImageJ built-in function (edit/selection/create selection) and the area quantified using the measure function (process/measure) to give total tumor (A_{T}) and IDO-expressing (A_{I}) areas. To calculate the percentage of A_{T} that overlapped with A_{I}, the selection of IDO was applied to the mask of PSMA, filled white, and the remaining tissue area measured as described above. The process was repeated for PSMA over IDO. To calculate the IDO⁺ area within tumor (A_{T+I}), the area of tumor without IDO (A_{T-I}) was subtracted from the total area of tumor (A_{T}). A_{T+I} was divided by A_{T} to give the percentage of total tumor area positive for IDO. A further description and examples of this analysis are provided in FIG. 5.

ELISPOT:

ELISPOT for measuring IFNγ release was performed as previously described (21). For these analyses, cryopreserved PBMC were thawed, and cultured with PAP protein antigen (Fitzgerald Industries, Acton, Mass.), media alone, or phytohemaglutinin (PHA). Stimulations were also conducted in the presence of IDO enzyme (5 ug/mL) or 1-MDT (2 mM). After 48 hours, plates were developed and spots enumerated using an automated ELISPOT reader (ImmunoSpot, CTL, Shaker Heights, Ohio).

Statistical Analysis:

Comparison of medians was made using T-tests or Wilkoxon ranked sum tests as indicated. Multiple comparisons across groups were made by Kruskal-Wallace test, with Dunn's correction for multiple comparisons. Correlations between linear variables parameter were made using a Pearson correlation coefficient. Analyses were conducted using GraphPad Prism software (version 5.01). For all analyses, a p value≤0.05 was considered statistically significant.

Results:

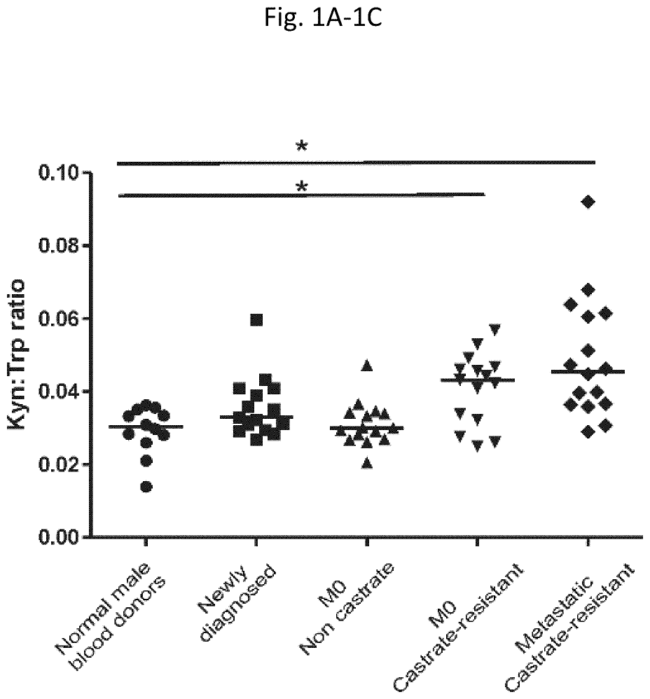

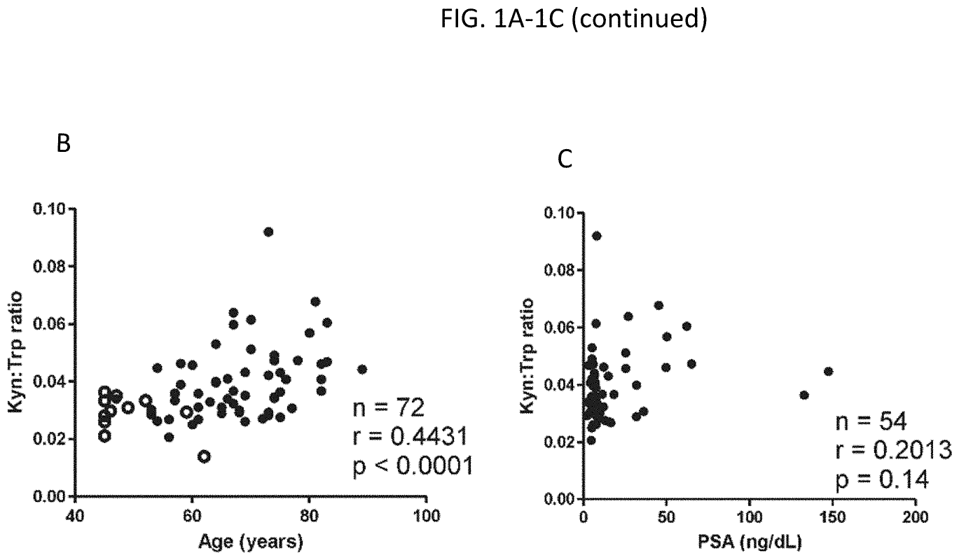

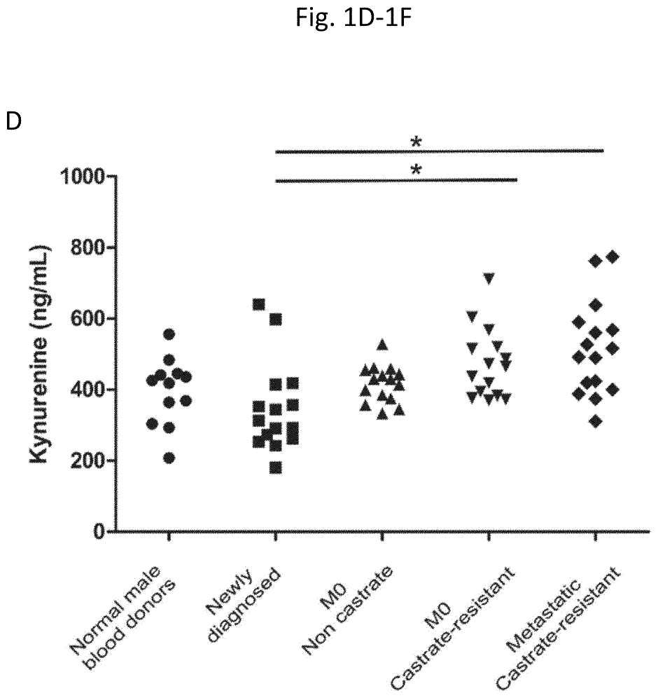

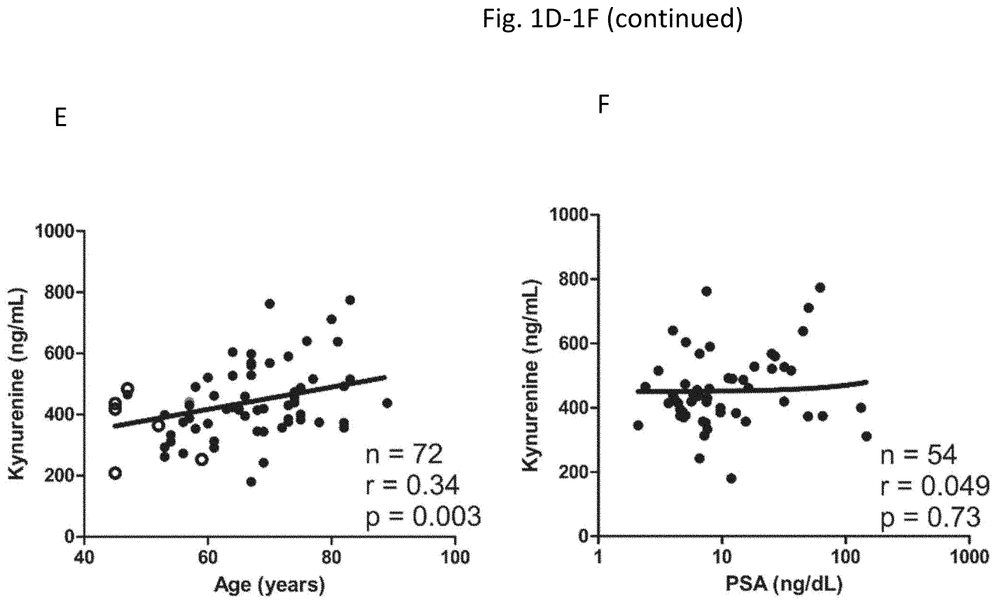

Patients with advanced prostate cancer have higher IDO activity. Sera samples from male volunteer blood donors without prostate cancer (n=12, median age 47, range 45-62), patients with newly diagnosed prostate cancer (n=14, median age 64, range 53-82), patients with non-metastatic, non-castrate, PSA-recurrent prostate cancer (n=15, median age 68, range 53-74), patients with non-metastatic, castration-resistant, PSA-recurrent prostate cancer (n=15, median age 73, range 60-89), and patients with castration-resistant, metastatic prostate cancer (n=16, median age 70, range 54-83) were evaluated for tryptophan and kynurenine concentrations as an assessment of IDO activity. The samples from patients with metastatic, castration-resistant prostate cancer (mCRPC) were those obtained at baseline from a clinical trial in which they subsequently received tumor-targeted vaccination and PD-1 blockade (17). As shown in FIG. 1A, the kynurenine-to-tryptophan (kyn:trp) ratio was generally higher in patients with prostate cancer compared with male volunteer blood donors, and highest in patients with more advanced stage of disease. However, because prostate cancer is an age-associated disease, kyn:trp ratios were assessed with respect to age as well as to the corresponding serum PSA level. As shown in FIG. 1B and FIG. 1C, kyn:trp was more associated with age than tumor volume, using serum PSA as a general assessment of tumor volume. Similar results were found for kynurenine concentrations directly (FIGS. 1D-1F).

Patients with prostate cancer treated with an anti-tumor vaccine and/or PD-1 develop increased IDO activity and expression. We have previously demonstrated in mice that vaccination targeting a tumor-associated antigen, with induction of IFNγ-secreting T cells specific for the antigen, elicits PD-L1 expression on tumor cells (14). We similarly found that PD-L1 expression increased on circulating tumor cells following vaccination of patients with prostate cancer using either sipuleucel-T or a DNA vaccine, both targeting the PAP prostate tumor antigen (15). Because IDO is also an IFNγ-regulated gene, we questioned whether prostate cancer immunotherapy similarly elicited increases in IDO expression. We have recently reported the results of a trial in which patients with mCRPC were treated with a DNA vaccine encoding PAP alone for 12 weeks, followed by pembrolizumab over the subsequent 12 weeks, or were treated with both agents for 12 weeks (17). Sera was assessed for kynurenine and tryptophan concentrations at baseline and at 12 and 24 weeks, effectively permitting an analysis of changes in IDO activity following treatment with vaccine alone, pembrolizumab alone, or the combination. As shown in FIG. 2A-2F, kyn:trp ratios generally increased pre-treatment to post-treatment over 12 weeks in patients treated with pembrolizumab alone (n=8, FIG. 2A, median 0.061 to 0.066, p=0.12), vaccine alone (n=10, FIG. 2B, median 0.049 to 0.053, p=0.098), and less with both concurrently (n=6, FIG. 2C, median 0.038 to 0.044, p=0.26). Kyn:trp ratios increased primarily in patients who did not experience a PSA decline during the 12-week period of treatment (n=17, FIG. 2E, median 0.053 to 0.061, p=0.007), compared with those who did experience a PSA decline during the 12-week period of treatment (n=7, FIG. 2D, median 0.037 to 0.046, p=0.88). IFNγ concentration was also directly evaluated in sera samples. As shown in FIG. 2F, IFNγ concentration was correlated to kyn:trp ratios determined from the same sera samples.

Biopsies were collected from individual metastatic lesions in nine patients at baseline and after 12 weeks who had received either vaccine alone, or a combination of vaccine and pembrolizumab. As shown in FIG. 3A and FIG. 3B, IDO staining within tumors was detectable and predominantly in the extracellular matrix in close proximity to IDO+ cells. Quantification of IDO staining within tumor regions demonstrated a significant increase after treatment in matched biopsies obtained from 8 patients (FIG. 3C, p=0.02, Wilcoxon signed rank test). The majority of cells staining positive were of the myeloid/macrophage lineage (CD163+), not prostate tumor cells (PSMA+), as shown in FIG. 3D. Increased IDO staining in tumors was generally associated with higher serum kyn:trp ratios (FIG. 3E, n=8, p=0.12).

IDO activity is associated with modest decrease in vaccine antigen-specific T-cell function. The observation that increased kyn:trp ratios were associated with absence of PSA declines suggested that increased IDO expression might be a mechanism of tumor resistance to antigen-specific T cells elicited with vaccination. To test this, peripheral blood cells obtained from 22 patients after treatment with vaccine and pembrolizumab were evaluated for PAP antigen-specific IFNγ release in the presence or absence of the IDO enzyme or 1-methyltrptophan, an IDO inhibitor that has been demonstrated to enhance T-cell activation in vitro (22). As shown in FIGS. 4A-4B, IDO enzyme tended to suppress the detection of PAP-specific IFNγ-secreting cells by ELISPOT, and similarly IDO inhibition increased the detection of IFNγ-secreting PAP-specific cells, although neither of these changes were statistically significant.

Discussion:

We observed that kyn:trp ratios were increased in patients with advanced prostate cancer. Because prostate cancer is a disease associated with more advanced age, we evaluated kyn:trp ratios with respect to age. Kyn:trp ratios were highly associated with patient age, and less associated with overall tumor burden, at least as measured by serum PSA. However, increased IDO activity did not appear to be independent of prostate cancer, because kyn:trp ratios were markedly induced, and to higher levels, following only 12 weeks of immunotherapy treatment with either a tumor vaccine or pembrolizumab, an effect independent of patient age or tumor volume. Our findings are consistent with previous reports demonstrating increased IDO gene expression in human prostate tumors relative to benign prostate tissue (10). Studies in TRAMP mice similarly showed expression of IDO in prostate tumors, and found that genetic crosses leading to the disruption of IDO activity delayed the development of prostate tumors (9). Together, these results, with our immunohistochemistry findings, suggest that IDO is expressed in the prostate tumor microenvironment, and that expression may be associated with disease progression, but that the expression may be most influenced by the presence of a T-cell immune response to the tumor. Of note, different cell types, including myeloid cells, can express IDO. Our studies suggest that the majority of the IDO expression within prostate tumors comes from CD163+ cells, notably cells of myeloid lineage including myeloid-derived suppressor cells (MDSC) and M2 macrophages. Notwithstanding, it is conceivable that IDO is produced by other cells, including tumor cells, and is taken up by these phagocytic cells.

IDO expression by human cells is known to be induced by IFNγ (18). In a recent report by Banzola and colleagues, the investigators evaluated prostate tumor cell lines and primary prostate cancer cells. They observed that IDO gene expression was higher in prostate cancer cells compared to benign tissues, associated with the expression of IFNγ and its receptors, and inducible in prostate cancer cell lines following IFNγ stimulation. Moreover, higher expression was associated with higher risk of biochemical recurrence following primary treatment (12). Our results demonstrate that immunotherapy treatment of prostate cancer, by either anti-tumor vaccine or PD-1 blockade, can increase IDO expression and activity. These effects are likely mediated by IFNγ released by lymphocytes activated by these treatments, consistent with our findings that serum IFNγ concentrations were highly associated with IDO activity, although this could not be definitively assessed. These findings are also consistent with our previous studies demonstrating that PD-L1, another protein induced following IFNγ exposure, is similarly expressed at higher levels following therapeutic anti-tumor vaccination in both murine models and in patients following treatment with this same PAP DNA vaccine (14, 15).

The finding that IDO expression is specifically increased following treatments that activate tumor-specific CD8+ T cells, notably by either vaccination or PD-1 blockade, suggests that it is a mechanism of immune evasion used by prostate cancer. This is further suggested by our finding that IDO expression was most induced in patients who did not experience evidence of anti-tumor response, as measured by any PSA decline, with immunotherapy treatment, whereas IDO activity was stable or decreased in patients with evidence of PSA decline. Curiously, some patients treated with combination therapy, previously demonstrated to elicit CD8+ T cell infiltration into tumors, did not have detectable increases in serum IDO activity. We suspect this was due to decreased tumor volume that was observed in these individuals treated with the combination (17). However, it is also possible that this was due to patient-specific pretreatment difference in their ability to mount IDO expression or due to differences in tumor volume or myeloid cell infiltrates, either of which could affect IDO activity and tumor response. Finally, we demonstrated that the tumor-specific T cells elicited with antigen-specific vaccination had decreased activity associated with IDO, as measured by antigen-specific IFNγ secretion by T cells, as this could be reversed in the presence of 1-methyltryptophan. These findings suggest that T-cell directed immunotherapy might be improved in the presence of IDO inhibition, or treatments aimed at reducing myeloid cells that may be producing IDO. Other preclinical studies have demonstrated that IDO activity is a mechanism of resistance to T-cell checkpoint blockade (7, 23). However, a recent clinical trial in patients with melanoma did not show any benefit to using an IDO inhibitor with PD-1 blockade (24). The use of these agents with anti-tumor vaccination, an approach that can lead to increased number of T cells secreting IFNy, however, should be explored. Of note, one phase II trial has evaluated 1-methyltryptophan (indoximod) following treatment with the prostate cancer vaccine sipuleucel-T, however final results from that trial are pending (25). Future studies will explore this approach directly in animal models and other human clinical vaccine trials.

Conclusions:

We showed that IDO activity is increased in patients with more advanced prostate cancer and this activity is augmented following prostate tumor-directed immunotherapy. This was detected both systemically, by evaluating kynurenine and tryptophan concentrations in the peripheral blood, and also by evaluating for IDO expression in prostate tumor tissues. The observation that expression was increased primarily in patients who did not have evidence of anti-tumor effect, and that IDO activity decreases the effector function of vaccine-induced T cells, suggests that it is a specific mechanism of immune resistance in prostate cancer. Together, these findings suggest that IDO inhibition should be explored in combination with immunotherapies targeting prostate cancer.

### REFERENCES

1. Hanahan D, Weinberg R A (2011) Hallmarks of cancer: the next generation. Cell. 144: 646-74. doi: 10.1016/j.cell.2011.02.013
2. Uyttenhove C, Pilotte L, Theate I, Stroobant V, Colau D, Parmentier N, Boon T, Van den Eynde B J (2003) Evidence for a tumoral immune resistance mechanism based on tryptophan degradation by indoleamine 2,3-dioxygenase. Nature medicine. 9: 1269-74. doi: 10.1038/nm934
3. Croitoru-Lamoury J, Lamoury F M, Caristo M, Suzuki K, Walker D, Takikawa O, Taylor R, Brew B J (2011) Interferon-gamma regulates the proliferation and differentiation of mesenchymal stem cells via activation of indoleamine 2,3 dioxygenase (IDO). PloS one. 6: e14698. doi: 10.1371/journal.pone.0014698
4. Mbongue J C, Nicholas D A, Torrez T W, Kim N S, Firek A F, Langridge W H (2015) The Role of Indoleamine 2, 3-Dioxygenase in Immune Suppression and Autoimmunity. Vaccines. 3: 703-29. doi: 10.3390/vaccines3030703
5. Munn D H, Mellor A L (2016) IDO in the Tumor Microenvironment: Inflammation, Counter-Regulation, and Tolerance. Trends in immunology. 37: 193-207. doi: 10.1016/j.it.2016.01.002
6. Holmgaard R B, Zamarin D, Li Y, Gasmi B, Munn D H, Allison J P, Merghoub T, Wolchok J D (2015) Tumor-Expressed IDO Recruits and Activates MDSCs in a Treg-Dependent Manner. Cell reports. 13: 412-24. doi: 10.1016/j.celrep.2015.08.077
7. Holmgaard R B, Zamarin D, Munn D H, Wolchok J D, Allison J P (2013) Indoleamine 2,3-dioxygenase is a critical resistance mechanism in antitumor T cell immunotherapy targeting CTLA-4. J Exp Med. 210: 1389-402. doi: 10.1084/jem.20130066
8. Komiya T, Huang C H (2018) Updates in the Clinical Development of Epacadostat and Other Indoleamine 2,3-Dioxygenase 1 Inhibitors (IDO1) for Human Cancers. Frontiers in oncology. 8: 423. doi: 10.3389/fonc.2018.00423
9. Kallberg E, Wikstrom P, Bergh A, Ivars F, Leanderson T (2010) Indoleamine 2,3-dioxygenase (IDO) activity influence tumor growth in the TRAMP prostate cancer model. Prostate. 70: 1461-70. doi: 10.1002/pros.21181
10. Feder-Mengus C, Wyler S, Hudolin T et al. (2008) High expression of indoleamine 2,3-dioxygenase gene in prostate cancer. Eur J Cancer. 44: 2266-75. doi: 10.1016/j.ejca.2008.05.023
11. Kolijn K, Verhoef E I, Smid M, Bottcher R, Jenster G W, Debets R, van Leenders G (2018) Epithelial-Mesenchymal Transition in Human Prostate Cancer Demonstrates Enhanced Immune Evasion Marked by IDO1 Expression. Cancer Res. 78: 4671-9. doi: 10.1158/0008-5472.CAN-17-3752
12. Banzola I, Mengus C, Wyler S et al. (2018) Expression of Indoleamine 2,3-Dioxygenase Induced by IFN-gamma and TNF-alpha as Potential Biomarker of Prostate Cancer Progression. Frontiers in immunology. 9: 1051. doi: 10.3389/fimmu.2018.01051
13. Rodriguez-Blanco G, Burgers P C, Dekker L J, Vredenbregt-van den Berg M S, Ijzermans J N, Schenk-Braat E A, Jenster G, Luider T M (2014) Serum kynurenine/tryptophan ratio is not a potential marker for detecting prostate cancer. Clinical biochemistry. 47: 1347-8. doi: 10.1016/j.clinbiochem.2014.05.001
14. Rekoske B T, Smith H A, Olson B M, Maricque B B, McNeel D G (2015) PD-1 or PD-L1 Blockade Restores Antitumor Efficacy Following SSX2 Epitope-Modified DNA Vaccine Immunization. Cancer immunology research. 3: 946-55. doi: 10.1158/2326-6066.CIR-14-0206
15. Rekoske B T, Olson B M, McNeel D G (2016) Antitumor vaccination of prostate cancer patients elicits PD-1/PD-L1 regulated antigen-specific immune responses. Oncoimmunology. 5: e1165377. doi: 10.1080/2162402X.2016.1165377
16. Zahm C D, Colluru V T, McNeel D G (2017) Vaccination with High-Affinity Epitopes Impairs Antitumor Efficacy by Increasing PD-1 Expression on CD8+ T Cells. Cancer immunology research. 5: 630-41. doi: 10.1158/2326-6066.CIR-16-0374
17. McNeel D G, Eickhoff J C, Wargowski E, Zahm C, Staab M J, Straus J, Liu G (2018) Concurrent, but not sequential, PD-1 blockade with a DNA vaccine elicits anti-tumor responses in patients with metastatic, castration-resistant prostate cancer. Oncotarget. 9: 25586-96. doi: 10.18632/oncotarget.25387
18. Werner-Felmayer G, Werner E R, Fuchs D, Hausen A, Reibnegger G, Wachter H (1991) Induction of indoleamine 2,3-dioxygenase in human cells in vitro. Advances in experimental medicine and biology. 294: 505-9.
19. Colluru V T, Zahm C D, McNeel D G (2016) Mini-intronic plasmid vaccination elicits tolerant LAG3+ CD8+ T cells and inferior antitumor responses. Oncoimmunology. 5: e1223002. doi: 10.1080/2162402x.2016.1223002
20. Schindelin J, Arganda-Carreras I, Frise E et al. (2012) Fiji: an open-source platform for biological-image analysis. Nature methods. 9: 676-82. doi: 10.1038/nmeth.2019
21. McNeel D G, Becker J T, Eickhoff J C et al. (2014) Real-time immune monitoring to guide plasmid DNA vaccination schedule targeting prostatic acid phosphatase in patients with castration-resistant prostate cancer. Clin Cancer Res. 20: 3692-704. doi: 10.1158/1078-0432.ccr-14-0169
22. Hou D Y, Muller A J, Sharma M D, DuHadaway J, Banerjee T, Johnson M, Mellor A L, Prendergast G C, Munn D H (2007) Inhibition of indoleamine 2,3-dioxygenase in dendritic cells by stereoisomers of 1-methyl-tryptophan correlates with antitumor responses. Cancer Res. 67: 792-801. doi: 10.1158/0008-5472.CAN-06-2925
23. Brown Z J, Yu S J, Heinrich B et al. (2018) Indoleamine 2,3-dioxygenase provides adaptive resistance to immune checkpoint inhibitors in hepatocellular carcinoma. Cancer Immunol Immunother. doi: 10.1007/s00262-018-2190-4
24. Long G V, Dummer R, Hamid O et al. (2018) Epacadostat (E) plus pembrolizumab (P) versus pembrolizumab alone in patients (pts) with unresectable or metastatic melanoma: Results of the phase 3 ECHO-301/KEYNOTE-252 study. 2018 ASCO Annual Meeting. Abstract #108.
25. Jha G G, Gupta S, Tagawa S T, Koopmeiners J S, Vivek S, Dukdek A Z, Cooley S A, Blazar B R, Miller J S (2017) A phase II randomized, double-blind study of sipuleucel-T followed by IDO pathway inhibitor, indoximod, or placebo in the treatment of patients with metastatic castration resistant prostate cancer (mCRPC). 2017 ASCO Annual Meeting. pp. Abstract #3066

Each publication, patent, and patent publication mentioned in this disclosure is incorporated in reference herein in its entirety. The present invention is not intended to be limited to the foregoing examples, but encompasses all such modifications and variations as come within the scope of the appended claims.

1. A method of reducing or inhibiting proliferation of a prostate cancer cell, increase killing or inducing apoptosis of the prostate cancer cell in a subject having prostate cancer, the method comprising:
administering at least one indoleamine 2,3 dioxygenase (IDO) inhibitor, at least one PD-1 inhibitor, and a DNA vaccine against prostate cancer each in an amount effective, in combination, to reduce or inhibit growth of the prostate cancer cell, increase killing or induce apoptosis of the prostate cancer cell, or combinations thereof in the subject.

2. The method of claim 1, wherein the subject is selected by (a) obtaining a sample from the subject and (b) detecting an increased level of IDO within the sample from the subject as compared to a control, wherein the subject with an increased IDO level is selected to be administered the combination.

3. The method of claim 2, wherein the increased level of IDO is detected:
(a) in a tumor sample of the subject by immunohistochemical staining; or
(b) in serum of the subject by determining the kynurenine-to-tryptophan (kyn:trp) ratio within the serum sample.

4. The method of claim 1,wherein the at least one IDO inhibitor is selected from the group consisting of 1-methyltryptophan (1-MT), epacadostat (INCB24360), navoximod (GDC-0919), Indoximod (NLG-8189), INCB024360, BMS-986205, NLG919, PF-06840003, and 8-nitrotryptanthrin.

5. (canceled)

6. The method of claim 1, wherein the PD-1 inhibitor is an anti-PD-1 antibody.

7. The method of claim 1, wherein the DNA vaccine encodes a prostatic acid phosphatase (PAP).

8. The method of claim 1, wherein the IDO inhibitor, PD-1 inhibitor and DNA vaccine are administered at the same time.

9. The method of claim 1, wherein the subject is selected to receive the inhibitors and the vaccine after not exhibiting reduced or inhibited growth of the prostate cancer cell after the PD-1 inhibitor and the vaccine are administered without the IDO inhibitor.

10. The method of claim 1, wherein the tumor is defined by an increase in IDO expression within at least one cell in the tumor microenvironment after treatment with a DNA vaccine, a PD-1 inhibitor or a combination thereof as compared to an untreated control cell.

11. (canceled)

12. (canceled)

13. A method of treating a subject having prostate cancer, the method comprising:
administering at least one indoleamine 2,3 dioxygenase (IDO) inhibitor, at least one PD-1 inhibitor, and a DNA vaccine against prostate cancer each in an amount effective, in combination, to treat the prostate cancer in the subject.

14. The method of claim 13, wherein the subject is selected by (a) obtaining a sample from the subject and (b) detecting an increased level of IDO within the sample from the subject as compared to a control, wherein the subject with an increased IDO level is selected to be treated.

15. The method of claim 13, wherein the at least one IDO inhibitor is epacadostat (INCB24360) or 1-MT.

16. (canceled)

17. The method of claim 13, wherein the DNA vaccine encodes a prostatic acid phosphatase (PAP) or the androgen receptor ligand binding domain (AR-LBD).

18. (canceled)

19. The method of claim 13, wherein the PD-1 inhibitor is an anti-PD-1 antibody.

20. The method of claim 13, wherein the IDO inhibitor, PD-1 inhibitor and DNA vaccine are administered at the same time.

21. The method of claim 13, wherein the subject has prostate cancer that was non-responsive to treatment by a PD-1 inhibitor in combination with DNA vaccine.

22. The method of claim 13, wherein the prostate cancer is defined by an increase in IDO expression within the tumor microenvironment after treatment with a DNA vaccine, a PD-1 inhibitor or a combination thereof as compared to a untreated control.

23. (canceled)

24. (canceled)

25. A method of eliciting an immune response by a DNA vaccine against a cancer in a subject having received or receiving the DNA vaccine, the method comprising: administering to the subject at least one indoleamine 2,3 dioxygenase (IDO) inhibitor and at least one PD-1 inhibitor, each administered in an effective amount, in combination, such that when the DNA vaccine, the IDO inhibitor, and the PD-1 inhibitor are administered to the subject the immune response elicited is greater than the immune response elicited by the DNA vaccine alone.

26. The method of claim 25, wherein the immune response is a T cell response.

27. The method of claim 25, wherein the at least one IDO inhibitor is selected from the group consisting of 1-methyltryptophan (1-MT), epacadostat (INCB24360), navoximod (GDC-0919), Indoximod (NLG-8189), INCB024360, BMS-986205, NLG919, PF-06840003, and 8-nitrotryptanthrin.

28. The method of claim 25, wherein the DNA vaccine encodes prostatic acid phosphatase (PAP).

29. (canceled)

30. The method of claim 25, wherein the IDO inhibitor, PD-1 inhibitor and DNA vaccine are administered at the same time.

31. The method of claim 25, wherein the subject has prostate cancer that was non-responsive to treatment by a PD-1 inhibitor in combination with DNA vaccine.

32. The method of claim 25, wherein the prostate cancer is defined by an increase in IDO expression within the tumor microenvironment after treatment with a DNA vaccine, a PD-1 inhibitor or a combination thereof as compared to a untreated control cell.
